(19) United States
(12) Patent Application Publication
(10) Pub. No.: US 2009/0238850 A1
(43) Pub. Date: Sep. 24, 2009

(54) ANTIMICROBIAL MATERIALS

(76) Inventor: Bryan NMI Greener, North Yorkshire (GB)

Correspondence Address: DIANA HOUSTON;SMITH & NEPHEW, INC., 1450 BROOKS ROAD, MEMPHIS, TN, 38116, US

(21) Appl. No.: 12/162,418

(22) PCT Filed: Jan. 26, 2007
(86) PCT No.: PCT/GB2007/000279

§ 371 Date: Sep. 5, 2008

(30) Foreign Application Priority Data
Jan. 27, 2006 (GB) 0601687.7

(51) Int. Cl. A61K 33/38; A61P 31/04; B05D 3/00

(52) U.S. Cl. 424/409; 424/618; 424/619; 424/600; 427/2.1

(57) ABSTRACT

Materials, compositions, and medical devices for the treatment or prophylaxis of microbial, including bacterial, infections, comprising at least one water-insoluble ceramic compound and at least one metal species. Methods of making such materials, compositions and medical devices.

This invention relates to materials for the treatment or prophylaxis of microbial, including bacterial, infection, in particular antimicrobial silver species, to compositions comprising such materials, to medical devices comprising these materials or compositions, to processes for the provision of such materials, compositions and devices, and to a method for the treatment or prophylaxis of microbial, including bacterial, infections using such materials, compositions or devices.

The clinical antimicrobial activity and efficacy of silver and silver compounds is well known. The activity of such metal-based antimicrobial, including antibacterial, materials is due to the release of metal-based species which are soluble, often in water, and that are delivered to the area to be treated. For medical device applications, a profile of release spanning several days is preferred.

Metal-based materials for the treatment or prophylaxis of microbial, including bacterial, infection exhibit a range of profile of release. Thus, the delivery rate (solubilisation) of silver species from silver metal, for example into aqueous media, is very low indeed. To increase the rate of silver solubilisation, silver salts have been employed, for example silver nitrate treatment. However, silver nitrate is highly soluble in water, and for medical device applications spanning several days, immediate solubility is not desirable.

Silver sulfadiazine does not dissolve immediately in the topical biological environment in which it is applied and has a profile of release spanning several days. However, in these silver salts the presence of a counter ion effectively dilutes the quantity of silver that can be provided in a given mass of material (63.5% of the total mass is silver in silver nitrate, only 30.2% in silver sulfadiazine).

The in vitro antimicrobial efficacy of silver oxides has recently attracted commercial interest. Their efficacy can exceed that of other silver compounds, and the presence of a counter ion of low mass, such as O²⁻, results in less dilution of the quantity of silver that can be provided in a given mass of material.

However, antimicrobial, including antibacterial, silver oxides (and silver(I) salts) suffer from inherent structural instability and/or photosensitivity, and this leads to poor storage stability and poor device compatibility, limiting their medical exploitation.

A conventional approach to enhancing the stability and ensuring the antimicrobial/antibacterial activity of silver ions is complexation of individual silver ions with stabilising ligands, such as sulfadiazine. The ligands needed to generate the relevant silver complex and/or the process for their preparation are often complex and/or costly.

Another approach is to generate stabilised silver oxide particles on a substrate by electrochemical or chemical means (including vapour deposition in the presence of an oxygen source, e.g. O₂ or O₃).

It is known from U.S. Pat. No. 5,151,122 to complex silver ions in situ onto solid substrates such as phosphates. For example, phosphate particles may conveniently be added to silver (I) ions present in an aqueous solution. The product is then sintered to provide a three-dimensional antibacterial ceramic device comprising silver ions. An object of U.S. Pat. No. 5,151,122 is to provide an antibacterial ceramic material in which silver ions will not elute into any contacting medium whatsoever. As noted hereinbefore, for medical device applications, a profile of substantive release spanning several days is preferred.

It is desirable to provide a material for the treatment or prophylaxis of microbial, including bacterial, infection that overcomes the limitations of known antimicrobial, including antibacterial, materials, i.e. it has a profile of release spanning several days, its efficacy exceeds that of traditional metal species (e.g. silver(I) salts), the presence of a counter ion effectively dilutes the quantity of active metal species (e.g. silver species) that can be provided in a given mass of material relatively little and it is stable under normal ambient conditions.

It is also desirable to provide compositions and devices comprising these materials, processes for the provision of such materials, compositions and devices, and a method for the treatment or prophylaxis of microbial, including bacterial, infections using such materials, compositions or devices.

Known methods of manufacture of medical devices in which the active silver is present on or in a surface of the device, such as topical dressings for the management of wounds, including surgical, acute and chronic wounds, and burns, and implants including long-term implants, such as artificial joints, fixation devices, sutures, pins or screws, catheters, stents and drains, suffer from the disadvantage that running a single manufacturing line for silver and non-silver products requires extended periods of down-time for cleaning.

It is therefore desirable to provide a process for the manufacture of such devices, in which the incorporation of silver metal or silver compounds is the final process step.

According to a first aspect of the present invention there is provided a material for the treatment or prophylaxis of microbial, including bacterial, infections, comprising at least one water-insoluble ceramic compound and at least one metal species, wherein, in use, the material releases metal species when in contact with a medium.

The material of the first aspect may comprise a reaction product of the at least one water-insoluble ceramic compound and the at least one metal species.

The material of the first aspect may comprise a complex of at least one water-insoluble ceramic compound and at least one metal species together with the reaction product of the at least one ceramic compound and the at least one metal species.

According to a second aspect of the present invention there is provided a method of preparing a material for the treatment or prophylaxis of microbial, including bacterial, infections, comprising the steps of:
i) preparing a solution of a metal species;
ii) contacting a water-insoluble ceramic compound with the metal species solution;
iii) filtering off the material; and
iv) drying the material.

The method of the second aspect of the present invention may include one or more of steps i) to iv) undertaken in the presence of light.

The method of the second aspect of the present invention may include one or more of steps i) to iv) undertaken in the absence of light.

According to a third aspect of the present invention there is provided a material for the treatment or prophylaxis of microbial, including bacterial, infections obtainable by the method of the second aspect, wherein, in use, the material releases metal species when in contact with a medium.

Preferably, the medium of the first or third aspects of the present invention is an aqueous medium. The medium may be a biological fluid, for example serum and/or wound exudate.

Preferably, the material according to the first or third aspects of the present invention has a profile of release of metal species when in contact with a medium of one or more days, particularly several days.

According to a fourth aspect of the present invention there is provided a composition comprising a material according to the first or third aspects of the present invention.

The at least one water-insoluble ceramic compound may be selected from the group consisting of phosphates, carbonates, silicates, aluminates, borates, zeolites, bentonite and kaolin.

Preferably, the ceramic compound is a phosphate-based compound. The phosphate-based compound may be derivatised.

The at least one metal species may be a silver, copper, zinc, manganese, gold, iron, nickel, cobalt, cadmium or platinum species.

Preferably, the metal species is a silver species.

When used herein the term ‘metal species’ means any material that includes metal ions, such as metal salts. For example, silver species include silver nitrate, silver perchlorate, silver acetate, silver tetrafluoroborate, silver triflate, silver fluoride, silver oxide and silver hydroxide. Silver species include materials comprising silver and oxygen atoms where at least one of each atomic type is directly bonded to the other, thus including but not restricted to oxides and hydroxides. Such species are termed silver-oxo species herein.

When used herein the term ‘water-insoluble optionally derivatised phosphate-based compound’ means any water-insoluble material comprising one or more phosphate units, each of which is optionally substituted by one or more groups such as halo, e.g. fluoro or chloro, or hydroxyl.

When used herein the term ‘water-insoluble’ means any material that is insoluble, substantially insoluble or sparingly soluble in water or saline at temperatures in the range of 10 to 40° C. at near-neutral pH values.

When used herein the term ‘reaction product of the silver species and a water-insoluble optionally derivatised phosphate-based compound’ means any such material, but in particular a silver species, in which at least one oxygen atom of at least one phosphate unit is directly bonded to a silver species.

Preferably, the silver and/or reaction product species are present on the surface of the phosphate-based material, in particular in the form of particles, which provide a suitably stable molecular template on which to form silver-oxo species, including hydroxides and oxides. Effectively a coating of the silver species and/or a reaction product of the silver species and the phosphate-based compound is formed on the surface of the phosphate-based compound. Preferred phosphate-based compounds are species that are not complex and/or costly.

The materials of the first aspect of the present invention overcome the limitations of known antimicrobial, including antibacterial, materials. For example, they have a profile of release spanning several days.

The materials exhibit a range of profile of release and delivery rate of the relevant active species, for example into aqueous media. The material compositions and components can be tailored to generate specific desired release rates, for example in aqueous media. For example, this can be achieved by modifying the loading, atomic structure, and/or the chemical nature of the phosphate-based compound.

The quantity of silver that can be provided in a given mass of material is effectively controlled by the phosphate-based compound loading.

The silver phosphate-based compound materials of the present invention exhibit enhanced stability compared with that of silver oxides. Compositions comprising them can be stored for long periods (up to several years) at ambient temperature and pressure in traditional sterile packaging. The silver phosphate-based compound materials are not photo-sensitive when packaged in standard medical device wrapping materials.

The atomic percentage of silver atoms in the materials of the present invention may suitably be in the range 0.001-100%. Silver loadings exceeding 20 atomic % can be achieved.

Examples of suitable phosphate-based compounds include polyphosphates with more than one phosphate monomer moiety. Polyphosphates are able to exist as linear and branched polymeric chains and cyclic structures, and offer a 2-D and 3-D array of phosphates of inflexible geometry.

Examples of suitable phosphates/phosphate-based compounds include orthophosphates, monocalcium phosphates, octacalcium phosphates, dicalcium phosphate hydrate (brushite), dicalcium phosphate anhydrous (monetite), anhydrous tricalcium phosphates, whitlockite, tetracalcium phosphate, amorphous calcium phosphates, fluoroapatite, chloroapatite, hydroxyapatite, non-stoichiometric apatites, carbonate apatites and biologically-derived apatites, and in particular calcium phosphates, calcium hydrogen phosphates and apatites.

The generation of silver species upon the surface of the phosphate-based compound scaffold can be achieved by combination of a silver(I) ion source, conveniently a water-soluble silver(I) salt, and a phosphate-based compound.

This can be achieved by any means known to a skilled chemist. For example, the solid phosphate-based compound can be introduced into an aqueous solution of silver(I) salt, and then separated, for example by filtration, after a time period corresponding to the desired extent of reaction. This is an example of a template synthesis.

Suitable compositions of the fourth aspect of the present invention include liquids, gels and creams for topical or internal administration per se or as a component of topical dressings, containing, e.g. the relevant silver phosphate-based compound complex particles in dispersion in the fluid phase.

Examples include hydrogels and xerogels, e.g. cellulosic hydrogels, such as cross-linked carboxymethylcellulose hydrogels, for the management of wounds, including surgical, acute and chronic wounds, and burns.

Suitable compositions also include surface-sterilising compositions, in particular for implantable devices, including long-term implants, such as artificial joints, fixation devices, sutures, pins or screws, catheters, stents, drains and the like.

In a fifth aspect the present invention provides a medical device, comprising a material of the first aspect of the present invention or a composition of the fourth aspect of the present invention.

Suitable devices include dressings, including topical dressings for the management of wounds, including surgical, acute and chronic wounds, and burns;

implants including long-term implants, such as artificial joints, fixation devices, sutures, pins or screws, catheters, stents and drains;
artificial organs and scaffolds for tissue repair; and
hospital equipment, including, for example, operating tables.

Often the composition of the fourth aspect of the present invention is present as a coating on a surface of the medical device or a component thereof. The devices of this fifth aspect may be stored for long periods, up to several years, at ambient temperature and pressure in traditional sterile packaging.

Suitable manufacturing methods for such devices are known to those skilled in the art and include dipping, fluid or powder coating and attachment via an adhesive or powder coating or blasting.

According to a sixth aspect of the present invention there is provided a process of manufacture of a medical device according to the fifth aspect, comprising incorporating a material of the first aspect or a composition of the fourth aspect into a medical device.

The process of the sixth aspect may comprise:

a) forming a material by generating metal species on a surface of a ceramic compound scaffold;

b) optionally formulating the material into a composition; and

c) applying or incorporating the material or composition onto or into a medical device.

Preferably, the process of the sixth aspect comprises:

a) optionally formulating a ceramic compound scaffold into a composition,

b) applying or incorporating the ceramic compound scaffold or composition onto or into a medical device, and

c) generating metal species on a surface of the ceramic compound scaffold.

That is, in situ generation of the metal species-ceramic compound material as a final manufacturing step.

For example, generation of silver species upon the surface of a phosphate-based compound scaffold may involve the combination of a silver(I) ion source, conveniently a water-soluble silver(I) salt, and a phosphate-based compound.

In a seventh aspect the present invention provides a method for the treatment or prophylaxis of microbial, including bacterial, infections, comprising the use of a material of the first aspect of the present invention, a composition of the fourth aspect of the present invention, or a medical device of the fifth aspect of the present invention.

Such a method for the treatment or prophylaxis of microbial, including bacterial, infections is useful in particular for the management of wounds, including surgical, acute and chronic wounds, and burns.

The present invention is further illustrated by the following Examples:

### EXAMPLE 1

### Deposition of Silver Species Surface Layer onto Calcium Hydrogen Phosphate Dihydrate

Calcium hydrogen phosphate dihydrate (200 mg) was added to a solution of silver(I) nitrate (50 mg) made up in distilled water (5 ml). The white phosphate powder turned yellow immediately upon immersion and was left to stand for 10 minutes, by which time colour change has ceased. The bright yellow powder was separated by Buchner filtration and washed with copious distilled water before desiccation and storage in the absence of light.

### EXAMPLE 2

### Deposition of Silver Species Surface Layer onto Tricalcium Phosphate

Tri-calcium phosphate (200 mg) was added to a solution of silver(I) nitrate (50 mg) made up in distilled water (5 ml). The white phosphate powder turned yellow immediately upon immersion and was left to stand for 10 minutes, by which time colour change has ceased. The bright yellow powder was separated by Buchner filtration and washed with copious distilled water before desiccation and storage in the absence of light.

### EXAMPLE 3

### Deposition of Silver Species Surface Layer onto Whitlockite

Whitlockite (200 mg) was added to a solution of silver(I) nitrate (50 mg) made up in distilled water (5 ml). The white phosphate powder turned slowly yellow upon immersion and was left to stand for 1 hour, by which time colour change has ceased. The yellow powder was separated by Buchner filtration and washed with copious distilled water before desiccation and storage in the absence of light.

### EXAMPLE 4

### Deposition of Silver Species Surface Layer onto Beta-Tricalcium Phosphate

beta-tricalcium phosphate (200 mg) was added to a solution of silver(I) nitrate (50 mg) made up in distilled water (5 ml). The white phosphate powder turned slowly yellow upon immersion and was left to stand for 1 hour, by which time colour change has ceased. The yellow powder was separated by Buchner filtration and washed with copious distilled water before desiccation and storage in the absence of light.

### EXAMPLE 5

### Deposition of Silver Species Surface Layer onto Calcium Phosphate Monobasic

calcium phosphate monobasic (200 mg) was added to a solution of silver(I) nitrate (50 mg) made up in distilled water (5 ml). The white phosphate powder turned slowly yellow upon immersion and was left to stand for 1 hour, by which time colour change has ceased. The yellow powder was separated by Buchner filtration and washed with copious distilled water before desiccation and storage in the absence of light.

### EXAMPLE 6

### Deposition of Silver Species Surface Layer onto Calcium Phosphate Tribasic

calcium phosphate tribasic (200 mg) was added to a solution of silver(I) nitrate (50 mg) made up in distilled water (5 ml). The white phosphate powder turned slowly yellow upon immersion and was left to stand for 1 hour, by which time colour change has ceased.

The yellow powder was separated by Buchner filtration and washed with copious distilled water before desiccation and storage in the absence of light.

### EXAMPLE 7

### Deposition of Silver Species Surface Layer onto Beta-Tricalcium Phosphate Bone Void Filler

Beta-tricalcium phosphate-based bone void filler (JAX, Smith & Nephew Orthopaedics) (1 g) was added to a solution of silver(I) nitrate (100 mg) made up in distilled water (10 ml). The white phosphate-based constructs turned slowly yellow upon immersion and was left to stand for 1 hour, by which time colour change has ceased. The yellow constructs were separated from the solution and washed with copious distilled water before desiccation and storage in the absence of light.

### EXAMPLE 8

### Deposition of Silver Species Surface Layer onto Hydroxyapatite/Chitosan Composite Fibres

Hydroxyapatite/chitosan composite fibres with a 30% weight content of hydroxyapatite (200 mg) were immersed in a solution of silver(I) nitrate (50 mg) made up in distilled water (5 ml). The white fibres immediately turned yellow upon immersion and were left to stand for 5 hours, by which time colour change has ceased and the final colour was brown. The brown fibres were separated from the solution and washed with copious distilled water before desiccation and storage in the absence of light.

### EXAMPLE 9

### Antimicrobial Activity of Example 2

The powder produced in Example 2 was tested for antibacterial activity by zone of inhibition test:

Pseudomonas aeruginosa NCIMB 8626 and Staphylococcus aureus NCTC 10788 were harvested. Serial 1:10 dilutions were performed to give a final concentration of 10⁸ bacteria/ml. Further dilutions were made for an inoculum count, down to 10⁻⁸ bacteria/ml, with the number of bacteria/ml determined using the pour plate method.

Two large assay plates were then set up and 140 ml of Mueller-Hinton agar was added evenly to the large assay plates and allowed to dry (15 minutes). A further 140 ml of agar was seeded with the corresponding test organism and poured over the previous agar layer. Once the agar had set (15 minutes), the plate was dried at 37° C. for 30 minutes with the lid removed. 8 mm plugs were removed from the plate by biopsy punch.

In triplicate, 10 mg of the composition prepared in Example 2 was transferred by spatula into the plate wells.

The plates were then sealed and incubated at 37° C. for 24 hours. The size of the bacterial zone cleared was measured using a Vernier calliper gauge, triplicates were averaged. Zones exceeded 3 mm for both organisms.

### EXAMPLE 10

### Deposition of Silver Species Surface Layer onto Calcium Hydrogen Phosphate Dihydrate

Calcium hydrogen phosphate dihydrate (200 mg) was added to a solution of silver(I) perchlorate (50 mg) made up in distilled water (5 ml). The white phosphate powder turned yellow immediately upon immersion and was left to stand for 10 minutes, by which time colour change has ceased.

The bright yellow powder was separated by Buchner filtration and washed with copious distilled water before desiccation and storage in the absence of light.

### EXAMPLE 11

### Deposition of Silver Species Surface Layer onto Calcium Hydrogen Phosphate Dihydrate

Calcium hydrogen phosphate dihydrate (200 mg) was added to a solution of silver(I) acetate (50 mg) made up in distilled water (5 ml). The white phosphate powder turned yellow immediately upon immersion and was left to stand for 10 minutes, by which time colour change has ceased. The bright yellow powder was separated by Buchner filtration and washed with copious distilled water before desiccation and storage in the absence of light.

### EXAMPLE 12

### Deposition of Silver Species Surface Layer onto Calcium Hydrogen Phosphate Dihydrate

Calcium hydrogen phosphate dihydrate (200 mg) was added to a solution of silver(I) tetrafluoroborate (50 mg) made up in distilled water (5 ml).

The white phosphate powder turned yellow immediately upon immersion and was left to stand for 10 minutes, by which time colour change has ceased. The bright yellow powder was separated by Buchner filtration and washed with copious distilled water before desiccation and storage in the absence of light.

### EXAMPLE 13

### Deposition of Silver Species Surface Layer onto Calcium Hydrogen Phosphate Dihydrate

Calcium hydrogen phosphate dihydrate (200 mg) was added to a solution of silver(I) triflate (50 mg) made up in distilled water (5 ml). The white phosphate powder turned yellow immediately upon immersion and was left to stand for 10 minutes, by which time colour change has ceased. The bright yellow powder was separated by Buchner filtration and washed with copious distilled water before desiccation and storage in the absence of light.

### EXAMPLE 14

### Deposition of Silver Species Surface Layer onto Calcium Hydrogen Phosphate Dihydrate

Calcium hydrogen phosphate dihydrate (200 mg) was added to a solution of silver(I) fluoride (50 mg) made up in distilled water (5 ml). The white phosphate powder turned yellow immediately upon immersion and was left to stand for 10 minutes, by which time colour change has ceased.

The bright yellow powder was separated by Buchner filtration and washed with copious distilled water before desiccation and storage in the absence of light.

### EXAMPLE 15

### Deposition of Silver Species Surface Layer onto Hydroxyapatite

Hydroxyapatite-coated, titanium-beaded, dumb bell-shaped implants (8 mm diam×14 mm cylinders with end-flanges) were immersed for approximately 5 minutes in 1% w/v silver nitrate (Aldrich Chemical Co.) solution made up in distilled water. Low ambient light conditions were enforced throughout this reaction. The HA coating yellowed during this time period, indicating presentation of silver species upon the surface of the coating. The dumb bell was removed, rinsed with excess distilled water and sterilised with 70% ethanol before drying at 40° C. in air.

### EXAMPLE 16

### Antimicrobial Activity of Example 15

The implants produced in Example 15 were tested for antibacterial activity by zone of inhibition test. A control was processed in the manner of Example 15, but lacking the silver nitrate.

Silver-treated device and control were individually immersed in 5 ml Staphylococcus aureus culture suspension (1×10⁷ cfu/ml) in the well of a 6-well culture plate (BD 353046). The culture plate was incubated with movement (150 rpm) for 24 hours at 37° C. After this incubation, each dumb bell was washed with 5 ml phosphate-buffered saline solution and stained with live/dead stain (Molecular Probes) for 15 minutes. Bacterial growth on each device was assessed by confocal microscopy.

There was a significant difference in the ability of each device to inhibit bacterial growth on its surface. The control device was completely colonised while the silver-treated device was largely bacteria-free.

### EXAMPLE 17

### Deposition of Silver Species Surface Layer onto Dressing

A polyurethane foam (Allevyn, Smith & Nephew Medical Limited) was formulated to contain 5% w/w calcium hydrogen phosphate powder (Aldrich Chemical Co.). The foam was immersed in 1% w/v aqueous silver(I) nitrate solution. This procedure was carried out under low ambient lighting conditions.

The white foam turned yellow after several seconds and was removed when the colour change ceased (approximately 1 minute) and rinsed with copious distilled water under cycling compression. The resulting foam was dried at 30° C. for 48 hours in the absence of light. The foam was cut and packed in ambient lighting conditions and sterilised by gamma irradiation (44 KGy).

The combinations of silver salts with phosphate-based ceramics result in thermodynamically stable reaction products. Following examination of the crystal structures of the ceramics used and the crystal structures of the metal oxides of the metals used, it has been hypothesised (without in any way limiting the present invention) that the structures of silver oxides and ceramic phosphates offered the greatest potential for compatibility (oxide oxygen geometry in silver oxides having a good fit with oxygen geometry in ceramic phosphates). It has been conjected that silver ions are capable of substituting for calcium or sodium ions in ceramic phosphates with minimal disturbance of the surrounding ceramic phosphate architecture. Other metal species may have similar compatibility.

1. A material for the treatment or prophylaxis of microbial, including bacterial, infections, comprising at least one water-insoluble ceramic compound and at least one metal species, wherein, in use, the material releases metal species when in contact with a medium.

2. A material according to claim 1, wherein the profile of release of metal species when in contact with a medium is one or more days.

3. A material according to claim 1, wherein the medium is aqueous.

4. A material according to claim 1, wherein the ceramic compound is selected from the group consisting of phosphates, carbonates, silicates, aluminates, borates, zeolites, bentonite, and kaolin.

5. A material according to claim 1, wherein the ceramic compound is a phosphate-based compound.

6. A material according to claim 5, wherein the phosphate-based compound is a polyphosphate.

7. A material according to claim 5, wherein the phosphate-based compound is selected from the group consisting of orthophosphates, monocalcium phosphates, octacalcium phosphates, dicalcium phosphate hydrate, dicalcium phosphate anhydrous, anhydrous tricalcium phosphates, whitlockite, tetracalcium phosphate, amorphous calcium phosphates, fluoroapatite, chloroapatite, hydroxyapatite, non-stoichiometric apatites, carbonate apatites, biologically-derived apatites, calcium phosphates, calcium hydrogen phosphates, and apatites.

8. A material according to claim 5, wherein the phosphate-based compound is derivatised derivatized.

9. A material according to claim 8, wherein the derivatised derivatized phosphate-based compound comprises one or more phosphate units substituted by one or more species selected from the group consisting of fluoro, chloro, or hydroxyl species.

10. A material according to claim 1, wherein the metal species is selected from the group consisting of silver, copper, zinc, manganese, gold, iron, nickel, cobalt, cadmium, and platinum species.

11. A material according to claim 1, wherein the metal species is a silver species.

12. A material according to claim 11, wherein the silver species is selected from the group consisting of silver nitrate, silver perchlorate, silver acetate, silver tetrafluoroborate, silver triflate, silver fluoride, silver oxide, and silver hydroxide.

13. A material according to claim 11, wherein the ceramic compound is a phosphate-based compound, wherein the silver species forms a coating on the surface of the phosphate-based compound.

14. A method of preparing a material for the treatment or prophylaxis of microbial, including bacterial, infections, comprising the steps of:
i) preparing a solution of a metal species;
ii) contacting a water-insoluble ceramic compound with the metal species solution;
iii) filtering off the material; and
iv) drying the material.

15. A method according to claim 14, wherein one or more of steps i) to iv) are undertaken in the presence of light.

16. A method according to claim 14, wherein one or more of steps i) to iv) are undertaken in the absence of light.

17. A material for the treatment or prophylaxis of microbial, including bacterial, infections obtainable by the method of claim 14, wherein, in use, the material releases metal species when in contact with a medium.

18. A material according to claim 17, rein the profile of release of metal species when in contact with a medium is one or more days.

19. A composition for the treatment or prophylaxis of microbial, including bacterial, infections, comprising a material according to claim 1.

20. A composition according to claim 19, wherein the composition is in the form of a liquid, gel, or cream.

21. A composition according to claim 19, wherein the composition is in the form of a hydrogel or xerogel.

22. A medical device, comprising a material according to claim 1 or a composition comprising the material of claim 1.

23. A medical device according to claim 22, wherein the material or the composition forms a coating on at least part of the medical device.

24. A medical device according to claim 22, wherein the medical device is selected from the group consisting of dressings, implants, artificial organs, scaffolds for tissue repair, and hospital equipment.

25. (canceled)

26. A process for manufacturing a medical device, comprising:
a) forming a material by generating metal species on a surface of a ceramic compound scaffold;
b) optionally formulating the material into a composition; and
c) applying or incorporating the material or composition onto or into a medical device.

27. A process for manufacturing a medical device, comprising:
a) optionally formulating a ceramic compound scaffold into a composition,
b) applying or incorporating the ceramic compound scaffold or composition onto or into a medical device, and
c) generating metal species on a surface of the ceramic compound scaffold.

28. A method for the treatment or prophylaxis of microbial, including bacterial, infections, comprising treating a subject having or at risk for having a microbial infection with the material of claim 1 or with a composition or medical device comprising the material of claim 1.

29. (canceled)

30. (canceled)

31. (canceled)

32. (canceled)

33. (canceled)
